(19) United States
(12) Patent Application Publication
(10) Pub. No.: US 2025/0295703 A1
(43) Pub. Date: Sep. 25, 2025

(54) TREATMENT AGENT FOR HAIR DAMAGE

(71) Applicant: TOHOKU UNIVERSITY, Sendai-shi (JP)

(72) Inventors: Kuniyasu NIIZUMA, Sendai-shi (JP); Teiji TOMINAGA, Sendai-shi (JP)

(73) Assignee: TOHOKU UNIVERSITY, Sendai-shi (JP)

(21) Appl. No.: 18/612,441

(22) Filed: Mar. 21, 2024

(51) Int. Cl. A61K 35/545; A61K 8/98; A61P 17/14; A61Q 5/00

(52) U.S. Cl. CPC A61K 35/545; A61K 8/981; A61P 17/14; A61Q 5/002

(57) ABSTRACT

A cell product for treating hair damage, namely, gray hair, hair loss, thinning hair, and/or the like, in particular, gray hair, the cell product comprising an SSEA-3-positive pluripotent stem cell (Muse cell) derived from a mesenchymal tissue in a living body or a cultured mesenchymal cell.

### TECHNICAL FIELD

The present disclosure relates to a cell product for regenerative therapy, having clinical effectiveness. More specifically, the present disclosure relates to a treatment agent for hair damage, comprising a pluripotent stem cell.

### BACKGROUND ART

Human hair has a lifetime, and regeneration and loss of hair are repeated (hair cycle) throughout life. One hair cycle is divided to three phases including the anagen phase during which hair is made by regeneration of the lower portions of hair follicles, the catagen phase during which hair growth is stopped and the lower portions of hair follicles are shrunk, and the telogen phase. Hair follicle stem cells and pigment stem cells are present in the bulge regions of hair follicles, and the hair bud regions, and such two kinds of stem cells are simultaneously activated in the anagen phase. Progeny cells of hair follicle stem cells regenerate the lower portions of hair follicles to produce hair bulb parts, and at the same time progeny cells of pigment stem cells travel to hair bulb parts and differentiate into pigment cells. In hair bulb parts, progeny cells of hair follicle stem cells incorporate pigments made by pigment cells in the course of differentiation into hair, and hair pigmented is regenerated. Thus, hair follicle stem cells and pigment stem cells are simultaneously activated and cooperate in the course of regeneration of hair follicles in the anagen phase, and therefore human hair is formed.

However, when the hair cycle of hair, in particular, cooperation in the course of regeneration of hair follicles in the anagen phase is broken by various factors such as aging and thus hormonal imbalance, stress, and neurological diseases, hair damage such as gray hair, hair loss, or thinning hair occurs.

With respect to treatment of such hair damage, there have been already marketed, as therapeutic medicines for losing hair and thinning hair, finasteride (having an effect of suppressing production of dihydrosterone being a male hormone that inhibits the activity of 5α-reductase and promotes losing hair), minoxidil (a compound that has the vasodilatory action and that promotes blood circulation and promotes hair growth), and the like.

Although there have been many reports of components and the like for prevention or improvement of gray hair in the treatment of gray hair, all of such components obtained have not been sufficient in terms of effectiveness and safety, and coloring of hair with hair colors is currently mainly performed.

Therefore, to provide a treatment agent capable of solving hair damage, in particular, gray hair, and the like is also to solve an eternal human problem.

On the other hand, researches related to mechanisms of gray hair, in particular, hair damage by stem cell transplantation and the like have also been made according to the advance of recent researches of regenerative therapy (Non-patent Document 1: P. Rabbani, et al., Cell, 145, 941-955 (2011), Non-patent Document 2: S. Tanimura, et al., Cell Stem Cell., 2011 February 4; 8(2): 177-187).

There has also been reported a case of the change in color of hair by adipose-derived autologous mesenchymal stem cell transplantation (Non-patent Document 3: J. Thadani et al., Stem cell Biology and Research 2015, doi: 10.7243/2054-717X-2-3, http://www.hoajonline.com/journals/pdf/2054-717X-2-3.pdf).

However, there is also not yet found any treatment agent that has been confirmed safeness and effectiveness and is capable of solving hair damage, in particular, gray hair and the like, and treatment is expected to be realized.

It has been revealed according to researches by Izawa et al. that pluripotent stem cells (Multilineage-differentiating Stress Enduring cells; Muse cells) which are present in mesenchymal cell fractions, which can be obtained without any gene introduction or any induction operation by cytokines, and which express SSEA-3 (Stage-Specific Embryonic Antigen-3) as a surface antigen are responsible for the pluripotency of mesenchymal cell fractions and can be applied to disease treatment aimed at tissue regeneration (for example, Patent Document 1; Non-patent Documents 4 to 6). It is known that Muse cells can be obtained from bone marrow aspirates, adipose tissues (Non-patent Document 7: Ogura F et al. Stem Cells Dev., Nov. 20, 2013 (Epub) (published on Jan. 17, 2014)) and dermal connective tissues, and are also broadly present in tissues and connective tissues in organs.

Patent Document 2 has disclosed differentiation of Muse cells into functional melanocytes by use of a combination of certain factor and cytokine, and Patent Document 3 has disclosed Muse cells being effective for treatment of skin diseases such as epidermolysis bullosa, but there has not been clear for any treatment effect enabling hair damage, in particular, gray hair, and the like to be eliminated from a clinical viewpoint.

### PRIOR ART DOCUMENTS

### Patent Documents

Patent Document 1: Japanese Patent No. 5185443
Patent Document 2: WO 2014/163206
Patent Document 3: WO 2018/235834

### Non-Patent Documents

Non-patent Document 1: Rabbani P et al. Cell, 145, 941-955(2011) Non-patent Document 2: Tanimura S et al. Cell Stem Cell., 2011 February 4; 8(2): 177-187 Non-patent Document 3: Thadani J et al. Stem cell Biology and Research 2015, http://www.hoajonline.com/journals/pdf/2054-717X-2-3.pdf, doi: 10.7243/2054-717X-2-3 Non-patent Document 4: Kuroda Y et al. Proc Natl Acad Sci USA 2010; 107: 8639-8643.
Non-patent Document 5: Wakao S et al. Proc Natl Acad Sci USA 2011; 108: 9875-9880.
Non-patent Document 6: Kuroda Y et al. Nat Protc 2013; 8: 1391-1415.
Non-patent Document 7: Ogura F et al. Stem Cells Dev., Nov. 20, 2013 (Epub) (published on Jan. 17, 2014)

### SUMMARY OF THE INVENTION

The present disclosure relates to a cell product for regenerative therapy, having clinical effectiveness, and specifically, an object thereof is to provide a treatment agent for hair damage, namely, gray hair, hair loss, thinning hair, and/or the like, in particular, gray hair.

The present inventors have unexpectedly found in the course of a clinical trial of Muse cells, for patients with cerebral infarction, that Muse cells have an effect on treatment of hair damage, namely, gray hair, hair loss, thinning hair, and/or the like, in particular, gray hair, and thus have found that Muse cells can be used in a treatment agent for hair damage, namely, gray hair, hair loss, thinning hair, and/or the like, in particular, gray hair, thereby leading to completion of the present disclosure.

Accordingly, the present disclosure provides the following Items.

[1]A treatment agent for hair damage, comprising an SSEA-3-positive pluripotent stem cell derived from a mesenchymal tissue in a living body or a cultured mesenchymal cell.

[2] The treatment agent of Item [1], wherein the hair damage is gray hair, hair loss, or thinning hair.

[3] The treatment agent of Item [1] or [2], wherein the hair damage is gray hair.

[4] The treatment agent of any of Items [1] to [3], wherein the pluripotent stem cell is one having all of the following characteristics:
(i) having low or no telomerase activity;
(ii) capable of differentiating into any of tridermic cells;
(iii) showing no neoplastic proliferation; and
(iv) having self-renewal capacities.

[5] The treatment agent of any of Items [1] to [4], wherein the pluripotent stem cell is one having all of the following characteristics:
(i) SSEA-3-positive;
(ii) CD105-positive;
(iii) having low or no telomerase activity;
(iv) capable of differentiating into any of tridermic cells;
(v) showing no neoplastic proliferation; and
(vi) having self-renewal capacities.

### Effect of the Invention

According to the present disclosure, Muse cell administration to a patient with hair damage, namely, gray hair, hair loss, thinning hair, and/or the like, in particular, gray hair can allow for treatment of hair damage, namely, gray hair, hair loss, thinning hair, and/or the like, in particular, gray hair.

While there is no specified mechanism on the present effect of Muse cells, it is considered from previous findings about Muse cells that, also in the case of hair damage, Muse cells can be efficiently migrated and engrafted to a disorder site where normal cooperation in the course of regeneration of hair follicles in the anagen phase is broken and Muse cells can be spontaneously differentiated at a site where Muse cells are engrafted, and thus there is no need for induction of differentiation to cells to be treated prior to transplantation. Muse cells are non-tumorigenic and also excellent in safety. Furthermore, Muse cells do not induce any immune rejection, and thus treatment with allogenic preparations produced from donors is also possible. Therefore, Muse cells having excellent characteristics as described above can provide clinically useful means for treatment of hair damage, namely, gray hair, hair loss, thinning hair, and/or the like, in particular, gray hair.

### EMBODIMENTS FOR CARRYING OUT THE INVENTION

### <1> Cell Product Comprising Muse Cell

The present disclosure relates to a treatment agent for hair damage, containing an SSEA-3-positive pluripotent stem cell (Muse cell). As used herein, the “treatment” encompasses healing, alleviating, ameliorating, preventing, and the like of symptoms, and the “treatment agent” means a preparation for use in the procedure, for example, the healing, alleviating, ameliorating, or preventing. The present disclosure is described below in detail.

### 1. Indications

The cell product comprising an SSEA-3-positive pluripotent stem cell (Muse cell) of the present disclosure is used for treatment of hair damage.

As used herein, the “hair damage” encompasses, for example, the change in color of hair, for example, graying hair, the change in quality of hair, for example, decrease in glow and hair fining, and the change in amount of hair, for example, losing hair and thinning hair.

Therefore, the treatment agent for hair damage of the present disclosure is used for healing, alleviating, ameliorating, or preventing of hair damage as described above.

The treatment of hair damage in the present disclosure is treatment of damage described above, occurring in the case of breakage of cooperation in the course of regeneration of hair follicles in the hair cycle, in particular, in the anagen phase, preferably encompasses treatment of, for example, gray hair, hair loss, and thinning hair, and is particularly preferably treatment of gray hair.

### 2. Cell Product

### (1) Pluripotent Stem Cell (Muse Cell)

The pluripotent stem cell to be used in the cell product of the present disclosure is a cell that has been found in a human living body and named “Muse (Multilineage-differentiating Stress Enduring) cell” by Dezawa et al. It is known that Muse cells can be obtained from bone marrow aspirates, adipose tissues (Non-patent Document 7: Ogura F et al. Stem Cells Dev., Nov. 20, 2013 (Epub) (published on Jan. 17, 2014)), and dermal connective tissues, and are also broadly present in tissues and connective tissues in organs. This cell also has both characteristics of pluripotent stem cell and mesenchymal stem cell and is identified as, for example, a cell positive for “SSEA-3 (Stage-specific embryonic antigen-3)” as a cell surface marker, preferably as a double-positive cell that is SSEA-3-positive and CD-105-positive. Therefore, Muse cells or a cell population containing Muse cells can be isolated from living tissues using, for example, expression of SSEA-3 only or a combination of SSEA-3 and CD-105 as an index. Methods for separation and identification of, and characteristics of Muse cells have been disclosed in WO2011/007900. Taking advantage of the high resistance of Muse cells to various external stresses, Muse cells can be selectively enriched by culturing the cells under various external stress conditions, such as under protease treatment, under hypoxic conditions, under low phosphate conditions, in a low serum concentration, under undernutrition conditions, under heat shock exposure, in the presence of toxic substances, in the presence of reactive oxygen species, under mechanical stimulation, and under pressure treatment. As used herein, pluripotent stem cells prepared from mesenchymal tissues in a living body or cultured mesenchymal tissues using SSEA-3 as an index (Muse cells), or a cell population comprising Muse cells, in a cell product for treatment of hair damage, may be simply referred to as “SSEA-3-positive cell.” As used herein, “non-Muse cells” may refer to cells included in mesenchymal tissues in a living body or cultured mesenchymal cells, other than “SSEA-3-positive cell”.

Muse cells or a cell population comprising Muse cells can be prepared from living tissues (e.g., mesenchymal tissues) using cell surface markers, SSEA-3, or SSEA-3 and CD-105, as indexes. As used herein, the term “living” body means mammal living body. In the present disclosure, living bodies exclude fertilized egg and embryos in developmental stages before blastula stage, but include embryos in developmental stages of blastula stage or later, including fetus and blastula. Examples of the mammal include, but not limited to, primates such as human and monkey; rodents such as mouse, rat, rabbit, and guinea pig; and cat, dog, sheep, pig, cattle, horse, donkey, goat, and ferret. Muse cells to be used in the cell product of the present disclosure are directly isolated from living tissues using markers, and thus are clearly distinguished from embryonic stem cells (ES cells) and induced pluripotent stem (iPS) cells. The term “mesenchymal tissue” refers to tissue such as bone, synovial membrane, fat, blood, bone marrow, skeletal muscle, dermis, ligament, tendon, dental pulp, umbilical cord, cord blood, and amnion, as well as tissues present in various organs. For example, Muse cells can be obtained from bone marrow, skin, adipose tissues, blood, dental pulp, umbilical cord, cord blood, or amnion. For example, and preferably, a mesenchymal tissue in a living body is collected, and then Muse cells are prepared from the tissue and used. Alternatively, using the preparation method described above, Muse cells may be prepared from cultured mesenchymal cells such as fibroblasts or bone marrow mesenchymal stem cells.

The cell population containing Muse cells to be used in the cell product of the present disclosure can also be prepared by a method comprising stimulating a mesenchymal tissue in a living body or cultured mesenchymal cells with an external stress to selectively increase cells that are resistant to the external stress, and collecting the cells with an increased abundance ratio.

The external stress may be any one of or a combination of the following: protease treatment, culturing under low oxygen concentration, culturing under low phosphate conditions, culturing under low serum concentration, culturing undernutrition conditions, culturing under heat shock exposure, culturing at low temperatures, freezing treatment, culturing in the presence of toxic substances, culturing in the presence of reactive oxygen species, culturing under mechanical stimulation, culturing under shaking, culturing under pressure treatment or physical shocks.

The protease treatment is preferably carried out for 0.5 to 36 hours in total to exert an external stress. The concentration of the protease is preferably used when cells adhered to a culture vessel are peeled off, when cell aggregates are separated into single cells, or when single cells are collected from a tissue.

Preferably, the protease is a serine protease, an aspartic protease, a cysteine protease, a metalloprotease, a glutamic protease, or an N-terminal threonine protease. More preferably, the protease is trypsin, collagenase, or Dispase.

Muse cells to be used in the cell product of the present disclosure may be autologous or allogeneic to a recipient who will receive the cells.

As described above, Muse cells or a cell population comprising Muse cells can be prepared from living tissues, for example, by using SSEA-3 positivity or SSEA-3 and CD-105 double positivity as an index, and human adult skin is known to comprise various types of stem cells and progenitor cells. However, Muse cells are not the same as these cells. These stem cells and progenitor cells include skin-derived progenitor cells (SKP), neural crest stem cells (NCSC), melanoblasts (MB), pericytes (PC), endothelial progenitor cells (EP), and adipose-derived stem cells (ADSC). Muse cells can be prepared using “non-expression” of markers unique to these cells as an index. More specifically, Muse cells can be isolated using as an index non-expression of at least one, e.g., 2, 3, 4, 5, 6, 7, 8, 9, 10, or 11, of 11 markers selected from the group consisting of CD34 (a marker for EP and ADSC), CD117 (c-kit) (a marker for MB), CD146 (a marker for PC and ADSC), CD271 (NGFR) (a marker for NCSC), NG2 (a marker for PC), vWF factor (von Willebrand factor) (a marker for EP), Sox10 (a marker for NCSC), Snail (a marker for SKP), Slug (a marker for SKP), Tyrp1 (a marker for MB), and Dct (a marker for MB). Muse cells can be prepared by using as an index, non-expression of, for example, without limitation, CD117 and CD146; CD117, CD146, NG2, CD34, vWF, and CD271; or the above-described 11 markers.

Muse cells to be used in the cell product of the present disclosure, having the above-described characteristics, may also have at least one selected from the group consisting of the following characteristics:
(i) having low or no telomerase activity;
(ii) capable of differentiating into any of tridermic cells;
(iii) showing no neoplastic proliferation; and
(iv) having self-renewal capacities
   Preferably, Muse cells to be used in the cell product of the present disclosure have all of the characteristics described above.

With respect to (i) above, the phrase “having low or no telomerase activity” means that the telomerase activity is low or undetectable when detected using, for example, TRAPEZE XL telomerase detection kit (Millipore Corporation). Having “low” telomerase activity means, for example, having a telomerase activity comparable to somatic human fibroblast, or having ⅕ or less telomerase activity, preferably 1/10 or less telomerase activity, as compared with that of HeLa cell.

With respect to (ii) above, Muse cells are capable of being differentiated into tridermic cells (endodermal, mesodermal, and ectodermal cells) in vitro and in vivo, and can be differentiated into, for example, hepatocytes (including cells expressing markers of hepatoblast or hepatocyte), neurons, skeletal muscle cells, smooth muscle cells, osteocytes, or adipocytes by in vitro inductive culturing. Muse cells may also show the ability to be differentiated into tridermic cells when transplanted in testis in vivo. Further, Muse cells are capable of migrating and engrafting to injured organs (such as heart, skin, spinal cord, liver, and muscle) when transplanted into a living body via intravenous injection and being differentiated into cells depending on the tissues.

With respect to (iii) above, Muse cells are characterized in that they proliferate at a growth rate of about 1.3 days and proliferate from a single cell in suspension culture to form embryoid body-like cell aggregates, and then arrest their proliferation after about 14 days when the aggregates reach a certain size. When these embryoid body-like cell aggregates are transferred to adherent culture, the cells restart proliferation and cells proliferated from the cell aggregates expand at a growth rate of about 1.3 days. Further, Muse cells are characterized in that, when transplanted into testis, they do not become cancerous for at least half a year.

With respect to (iv) above, Muse cells have self-renewal (self-replication) capacities. The term “self-renewal,” as used herein, means that the followings can be observed: differentiation into tridermic cells from cells contained in first embryoid body-like cell aggregates obtained by culturing single Muse cells in a suspension culture; formation of next-generation second embryoid body-like cell aggregates by again culturing single cells in the first embryoid body-like cell aggregates in a suspension culture, and differentiation again into tridermic cells and formation of third embryoid body-like cell aggregates in a suspension culture. Self-renewal may be repeated for one or more cycles.

### (2) Preparation and Use of Muse Cell-Containing Cell Product

The Muse cell-containing cell product of the present disclosure can obtained by, without limitation, suspending Muse cells or a cell population comprising Muse cells, obtained in (1) above, in a physiological saline or a suitable buffer solution (e.g., a phosphate buffered saline). In this case, when only a small number of Muse cells is isolated from an autologous or allogeneic tissue, the cells may be cultured before cell transplantation until the predetermined number of cells is attained. As there has been already reported (WO2011/007900), Muse cells are non-tumorigenic, and thus are less likely to be cancerous and thus are safe, even if such cells collected from a living tissue are contained in an undifferentiated state. The collected Muse cells can be cultured in any normal growth medium (e.g., alpha-minimum essential medium (α-MEM) supplemented with 10% calf serum), without particularly limitation. More specifically, with reference to the above-described WO2011/007900, Muse cells can be cultured and proliferated using appropriately selected culture medium, additives (e.g., antibiotics, and serum) and the like, to prepare a Muse cell-containing solution at a predetermined concentration. When the Muse cell-containing cell product of the present disclosure is administered to a human subject, bone marrow aspirate is collected from human ilium bone. For example, a bone marrow mesenchymal stem cell is cultured as an adherent cell obtained from the bone marrow aspirate and proliferated until reaching the cell amount where a therapeutically effective amount of Muse cells can be obtained. Thereafter, Muse cells are isolated using an antigen marker SSEA-3 as an index to prepare an autologous or allogeneic Muse cell-containing cell product. Alternatively, for example, a bone marrow mesenchymal stem cell obtained from the bone marrow aspirate can be cultured under external stress conditions, so that Muse cells can be grown and enriched until the amount thereof reaches a therapeutically effective amount, thereby preparing an autologous or allogeneic Muse cell-containing cell product.

When Muse cells are used in a cell product, the cell product may also comprise dimethyl sulfoxide (DMSO), serum albumin and the like for protection of the cells, and any antibiotic and the like for prevention of contamination and proliferation of bacteria. The cell product may further comprise other pharmaceutically acceptable components (e.g., carriers, excipients, disintegrants, buffer agents, emulsifiers, suspending agents, soothing agents, stabilizers, preservatives, antiseptics, physiological saline). These agents and drugs can be added to the cell product at appropriate concentrations by the skilled person. Thus, Muse cells can also be used in a pharmaceutical composition comprising various additives.

The number of Muse cells contained in the cell product prepared above can be appropriately adjusted to achieve desired effects on treatment of hair damage, in consideration of, for example, the sex, age, and weight of the subject, the condition of the affected area, and the condition of such cells to be used. Individuals as the subject include, but not limited to, mammals such as human. The Muse cell-containing cell product of the present disclosure may be administered in multiple times at appropriate intervals (e.g., twice a day, once a day, twice a week, once a week, once every two weeks, once a month, once every two months, once every three months, or once every six months) until the desired therapeutic effect is obtained. Therefore, the therapeutically effective amount is preferably, for example, 1 to 10 doses of 1×10³ to 1×10¹⁰ cells/individual/dose for a year, depending on the state of the subject. The total amount administered to an individual is, without limitation, 1×10³ cells to 1×10¹⁰ cells, preferably 1×10⁴ cells to 1×10¹⁰ cells, more preferably 1×10⁵ cells to 1×10⁹ cells.

Muse cells to be used in the cell product of the present disclosure are characterized in that these migrate and engraft to a disorder site. Thus, the site and method of administration of the cell product in administration of the cell product are not limited, and examples include intravascular administration (intravenous, intra-arterial), and topical administration. Preferred is intravascular administration (intravenous, intra-arterial), or topical administration to a hair damage site.

The Muse cell-containing cell product of the present disclosure allows for realization of clinical treatment of hair damage, namely, gray hair, hair loss, thinning hair, and/or the like, in particular, gray hair.

The present disclosure will be described in more detail with reference to examples below, but is not limited to the examples in any way.

### EXAMPLES

### <Preparation of Human Muse Cells>

Muse cells were obtained according to the method for isolation and identification of human Muse cells described in WO2011/007900. Such Muse cells were subjected to expansive enrichment culture with culture of mesenchymal stem cells under stress conditions.

<Clinical Trial of Muse Cells (CL2020) for Patient with Cerebral Infarction>

A clinical trial for patients with cerebral infarction was performed under the following conditions.
Name of disease: cerebral infarction
Overview of trial: to examine safety and effectiveness in intravenous administration of CL2020, for patients with cerebral infarction, with placebo as control.
Design of trial: placebo-control, randomization (assigned at a ratio of positive control 2.5:placebo 1), double-blind
Number of cases: 35 cases (positive control 25 cases, placebo 10 cases)
Age: 20 years old or older and younger than 80 years old
Gender: both
Eligibility criteria:
   Patient with cerebral infarction, having nervous symptoms such as physical dysfunction at the time point after lapse of the period of application of standard acute-phase treatment
   Patient within 14 days to 28 days after the onset of cerebral infarction
   Patient with a National Institutes of Health Stroke Scale (NIHSS) of 6 or greater upon registration, and the like
Exclusion criteria:
   Patient with a decreased level of consciousness observed
   Patient with hemorrhagic transformation observed by CT or MRI, and the like
Administration method: drip infusion of one bag of CL2020 (Muse cells: 1.5×10⁷ cells/about 52 ml) or placebo (about 52 ml) over 10 to 15 minutes.
Test period: 52 weeks
Observation/inspection items:
   12-Lead electrocardiogram
   Blood test, urine test
   Assessment of symptoms of cerebral infarction (NIHSS (National Institute of Health Stroke Score), mRS (modified Rankin Scale), SIAS (Stroke Impairment Assessment Set), BI (Barthel Index), FMMS (Fugl-Meyer Motor Scale), EQ-5D-5L (EuroQol-5-dimension 5 level))
   Others

### <Results of Clinical Trial>

Results of primary endpoint:

No severe adverse effect causing problems in the conduct of the clinical trial was observed with respect to the safety until 52 weeks after administration of CL2020, as the primary endpoint, and favorable tolerability could be confirmed.
Results of secondary endpoint (summary):
   Most patients had a modified Rankin Scale (mRS) of 4 or 5 before administration of placebo (bogus medicine) or CL2020.
   The lower limit of 95% confidence interval (21.1 to 61.3%) of the rate (responder rate) of a subject having a mRS of 2 or less after 12 weeks (3 months) from administration exceeded a threshold responder rate of 8.7% set in advance, and the treatment effect of CL2020 was confirmed. The responder rate at 12 weeks after administration reached 40% (10 cases/25 cases) in the CL2020-administration group, and exceeded by 30% as compared with 10% (1 case/10 cases) in the placebo-administration group (p=0.08). The responder rate at 52 weeks (one year) after administration reached 68.2% (15 cases/22 cases) in the CL2020-administration group, and a between-group difference of 30% or more, from that in the placebo-administration group (37.5%, 3 cases/8 cases), was kept.
   The subjects of 7 cases reached a mRS of 1 at 52 weeks in effectiveness evaluation until 52 weeks after administration and all the cases belonged to the CL2020-administration group (31.8%, 7 cases/22 cases), but such cases were not confirmed in the placebo-administration group.
   The National Institute of Health Stroke Score (NIHSS) serving as a severity evaluation index for neurological disorders includes 15 endpoints including consciousness, visual field, ocular motion, facial paralysis, extremity muscle strength, ataxia, sensation, language, and the like, and the total of scores of each endpoint is 0 to 42 points and a higher score means severer. While subjects of 0 cases had an NIHSS of 1 or less (almost normal state) before administration of placebo and CL2020, subjects of 5 cases were determined to have an NIHSS of 1 at 52 weeks (one year) and all the cases belonged to the CL2020-administration group (23.8%, 5 cases/21 cases).
   The respective transitions of the average values of the amounts of change in Fugl-Meyer Motor Scale (FMMS) as an exercise function evaluation index, from the baselines about the upper limbs and lower limbs, were compared. The exercise function improvement effect having a statistically significant difference (p<0.01) from the placebo-administration group was observed with respect to the upper limbs for a period of 4 weeks to 52 weeks after administration in the CL2020-administration group. The exercise function improvement effect of the lower limbs was observed in both groups.
Findings about hair/skin:

While blackening of hair, hair growth, improvement of hair glow, improvement in amount of hair, improvement of skin blemishes, and the like were observed as other findings in 7 cases in the clinical trial as shown below, all the cases belonged to the CL2020-administration group.

### <Case 1 (#006):>

70 years old, female, CL2020 administered on Dec. 10, 2018
Changed in hair at 28 weeks after administration (felt black for no special reason)
Told hair blackened from family member(s) and beautician(s) at 36 weeks after administration.
   (Physician finding) Gray hair at the time of hospital admission, but now strong black hair around the occipital region. Black hair is seen little by little also in the frontal region.
   (Dermatologic finding) Growth of black hair is observed around the occipital region.
   In addition, clear growth of black and short hair is observed at the boundary portion with the frontal region-forehead, where such hair growth in a middle-aged patient is usually considered difficult. Hair regeneration is considered to be sufficiently possible by pluripotent stem cells.
Hair is blackened, and blemishes on the face and blemishes on the left wrist also are increasingly lightened at 44 weeks after administration.
   (Physician finding) The color of hair clearly blackened and some part of hair has a gray tip and a black root. In particular, black hair is observed on the forehead and the circumference of the auricle. Vellus hair grown on the forehead which had been bald in an M-shaped manner. Blemishes on the left cheek portion and the left forearm seemed to be lightened.
   (Dermatologic finding) The color of hair clearly changed and hair seemed to be grown.
No change in color of hair at 52 weeks after administration (physician finding).
   (Evaluation of cerebral infarction symptoms at 52 weeks after administration: mRS=1, NIHSS=0)

### <Case 2 (#012):>

75 years old, male, CL2020 administered on Jan. 10, 2019
(Physician finding) The patient said that he had obtained black hair mainly in the occipital region and the eyebrow at about 11 weeks after administration, although the patient had gray hair at the time of hospital admission. What the patient said was confirmed to be certainly true.
Hair gradually blackened first in the occipital region at about 16 weeks after administration, at the time of hospital discharge, the circumference of the auricle (the circumference of so-called sideburns) then blackened, and front hair also blackened now. Hair seemed to be gradually blackened.
(Dermatologic finding) Black hair densely re-grown toward the occipital region from both the temporal regions at about 20 weeks after administration. The same finding was not confirmed toward the frontal region from the parietal region. Hair toward the occipital region from the temporal region clearly differed from that in the parietal region in terms of both color tone and properties. Possibly, tissues are newly regenerated.
   (Evaluation of Cerebral Infarction Symptoms at 52 Weeks after Administration: MRS=4, NIHSS=4)

### <Case 3 (#013):>

65 years old, male, CL2020 administered on Jan. 15, 2019
Told hair blackened from family member(s) and workplace member(s) at about 21 weeks after administration.
The color of hair seemed to be blackened at about 45 weeks after administration, than before.
Hair cut at about 52 weeks after administration, and hair clearly revealed to be blackened and grown.
   (Physician finding) The color of hair seemed to be blackened than before.
No dermatologic examination.
   (Evaluation of Cerebral Infarction Symptoms at 52 Weeks after Administration: MRS=1, NIHSS=2)

### <Case 4 (#017):>

78 years old, male, CL2020 administered on Feb. 19, 2019
Hair felt blackened clearly by the ward physician and the like at about 07 weeks after administration.
Heard that hair in the occipital region felt blackened by family member(s) at about 20 weeks after administration.
No dermatologic examination.
   (Evaluation of Cerebral Infarction Symptoms at 20 Weeks after Administration: MRS=3, NIHSS=4)
   (No Evaluation of Cerebral Infarction Symptoms at 52 Weeks in this Case Because of Stopping of the Trial on the Way)

### <Case 5 (#018):>

66 years old, female, CL2020 administered on Feb. 21, 2019
Hair significantly improved in glow at about 2 years after administration, not rendered loose, and also increased in amount. This is also told at a hair salon.
No dermatologic examination.
   (Evaluation of Cerebral Infarction Symptoms at 52 Weeks after Administration: MRS=2, NIHSS=2)
   (Evaluation of Cerebral Infarction Symptoms at 2 Years after Administration: MRS=2, NIHSS=4)

### <Case 6 (#027):>

69 years old, male, CL2020 administered on Jun. 13, 2019
(Physician finding) The color of hair clearly blackened at about 37 weeks after administration than before. Told hair clearly blackened particularly by family member(s) met for the first time in one and a half months.
   (Dermatologic finding) black hair bundle observed toward the occipital region from the parietal region. No loss of pigments, and the like, is observed with a dermatoscopy, and middle-aged hair is considered to be obtained.
   (Evaluation of Cerebral Infarction Symptoms at 36 Weeks after Administration: MRS=4, NIHSS=8)
   (Evaluation of Cerebral Infarction Symptoms at 52 Weeks after Administration: MRS=3, NIHSS=5)

### <Case 7 (#031):>

66 years old, male, CL2020 administered on Aug. 7, 2019
(Physician finding) The change in color of hair is confirmed by the physician on the day at about 52 weeks after administration. The color of hair clearly darker than that at the time of hospital admission.
   (Dermatologic finding) Black hair as head hair is observed to be regenerated, with being mixed with gray hair.
   (Evaluation of Cerebral Infarction Symptoms at 52 Weeks after Administration: MRS=4, NIHSS=5)

### INDUSTRIAL APPLICABILITY

The cell product of the present disclosure exhibits blackening of hair, hair growth, improvement of hair glow, improvement in amount of hair, and the like, in the results of the clinical trial, and can be applied to treatment of hair damage, namely, gray hair, hair loss, thinning hair, and/or the like, in particular, gray hair. The cell product can also be applied to cosmetic use, for example, regeneration/repair of the skin, because of exhibiting improvement of skin blemishes (the left cheek portion, the left forearm, and the like).

While the invention has been described in detail with reference to exemplary embodiments thereof, it will be apparent to one skilled in the art that various changes may be made, and equivalents employed, without departing from the scope of the invention. Each of the aforementioned documents as well as Japanese Patent Application No. 2021-148888 is incorporated by reference herein in its entirety.

What is claimed is:

1. A method for treatment for hair damage in a subject in need thereof, comprising administering an effective amount of an SSEA-3-positive pluripotent stem cell derived from a mesenchymal tissue in a living body or a cultured mesenchymal cell to said subject.

2. The method according to claim 1, wherein the hair damage is gray hair, hair loss, or thinning hair.

3. The method according to claim 1, wherein the hair damage is gray hair.

4. The method according to claim 1, wherein the pluripotent stem cell is one having all of the following characteristics:
(i) having low or no telomerase activity;
(ii) capable of differentiating into any of tridermic cells;
(iii) showing no neoplastic proliferation; and
(iv) having self-renewal capacities.

5. The method according to claim 1 wherein the pluripotent stem cell is one having all of the following characteristics:
(i) SSEA-3-positive;
(ii) CD105-positive;
(iii) having low or no telomerase activity;
(iv) capable of differentiating into any of tridermic cells;
(v) showing no neoplastic proliferation; and
(vi) having self-renewal capacities.
